(19) United States
(12) Patent Application Publication
(10) Pub. No.: US 2023/0372386 A1
(43) Pub. Date: Nov. 23, 2023

(54) Pet Food Compositions

(71) Applicant: Hill's Pet Nutrition, Inc., Topeka, KS (US)

(72) Inventor: Matthew JACKSON, Topeka, KS (US)

(21) Appl. No.: 18/321,563

(22) Filed: May 22, 2023

Related U.S. Application Data
(60) Provisional application No. 63/344,980, filed on May 23, 2022.

(51) Int. Cl. A61K 31/716; A23K 20/163; A23K 20/121; A23K 10/30; A23K 50/40; A61K 9/00; A61K 31/352; A61P 1/14; A61P 13/12

(52) U.S. Cl. CPC A61K 31/716; A23K 20/163; A23K 20/121; A23K 10/30; A23K 50/40; A61K 9/0056; A61K 31/352; A61P 1/14; A61P 13/12

(57) ABSTRACT

Pet food compositions and methods are provided herein. The pet food composition typically includes a gut microbiome component comprising: (i) β-1,3/1,6-glucan; (ii) polyphenolic isoflavonoids, wherein the pet food composition has a weight ratio of the β-1,3/1,6-glucan of (i) to the polyphenolic isoflavonoids of (ii) of about 1:1 to about 5:1, and wherein the gut microbiome component is present in an amount effective to produce, after about 3 days post-ingestion by a pet, an increase in the weight ratio of propionate to branched short chain fatty acids in the feces and an increase in the weight ratio of short chain fatty acids to branched short chain fatty acids in the feces of the pet.

### CROSS-REFERENCE TO RELATED PATENT APPLICATIONS

This application claims priority to U.S. Appl. No. 63/344,980, entitled “PET FOOD COMPOSITIONS,” filed on May 23, 2022; the contents of which are incorporated herein by reference in its entirety for all purposes.

### BACKGROUND

The well-being of domestic animals is closely related to their feeding. Correct feeding should result in a fit and healthy pet. To achieve correct feeding, one may utilize certain ingredients and concentrations of those ingredients which yield beneficial effects for the animal. Such beneficial effects may include protection against inflammation, kidney damage, renal insufficiency, cardiovascular disease, and/or high urine solute concentration.

Renal failure is one of the most common causes of death in dogs. In animals which suffer from renal disease, several blood indices are used to determine the severity of the disease. These indices include blood urea nitrogen (BUN) and creatinine. BUN and creatinine levels in the bloodstream increase during the course of renal failure because damage to the kidney of the animal makes the kidney inadequate to filter waste products. Because inadequate filtration of waste products is the fundamental basis of renal disease, BUN and creatinine are considered the primary indicators of renal disease.

Current dietary therapies to reduce measured BUN, creatinine, and phosphorus levels include decreasing the amount of dietary protein to levels where amino acids are present in insufficient quantities. Decreasing the level of dietary protein decreases BUN since urea is ultimately derived from protein. However, such diets may result in other problems developing for the animal as the animal's protein needs are unmet.

Accordingly, there remains a need for pet food compositions and methods for alleviating, reducing, or mitigating renal disease without potentially inducing other health problems in the pet.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to pet food compositions that increase the weight ratio of propionate to branched short chain fatty acids and, preferably, increase the weight ratio of short chain fatty acids to branched short chain fatty acids in the feces of a pet consuming such pet food composition. In accordance with one aspect of the invention, provided is a pet food composition having a gut microbiome component comprising: (i) β-1,3/1,6-glucan; (ii) polyphenolic isoflavonoids, wherein the pet food composition has a weight ratio of the β-1,3/1,6-glucan of (i) to the polyphenolic isoflavonoids of (ii) of about 1:1 to about 5:1, and wherein the gut microbiome component is present in an amount effective to produce, after about 3 days post-ingestion by a pet, an increase in the weight ratio of propionate to branched short chain fatty acids in the feces and an increase in the weight ratio of short chain fatty acids to branched short chain fatty acids in the feces of the pet.

In accordance with another aspect, provided is a pet food composition having a prebiotic component; and a gut microbiome component having (i) β-1,3/1,6-glucan and (ii) polyphenolic isoflavonoids, wherein pet food composition has a weight ratio of the β-1,3/1,6-glucan of (i) to the polyphenolic isoflavonoids of (ii) of about 1:1 to about 5:1. In some embodiments, the weight ratio of β-1,3/1,6-glucan to polyphenolic isoflavonoids is about 1:1 to about 3:1. Yet in further embodiments, the weight ratio of β-1,3/1,6-glucan to polyphenolic isoflavonoids is about 2:1.

The pet food compositions may preferably include β-1,3/1,6-glucan in an amount of from about 0.3 to about 5 wt. %, based on the total weight of the pet food composition. Additionally or alternatively, the pet food compositions may include polyphenolic isoflavonoids in an amount of from about 0.05 to about 0.5, based on the total weight of the pet food composition.

According to a further aspect, a method is provided for alleviating, mitigating, and/or reducing renal disease in a pet. The method typically includes administering an effective amount of a pet food composition disclosed herein to increase a weight ratio of propionate to branched short chain fatty acids in the feces and an increase in a weight ratio of short chain fatty acids to branched short chain fatty acids in the feces of the pet.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the pet food composition. The symbol “° ” refers to a degree, such as a temperature degree or a degree of an angle. The symbols “h”, “min”, “mL”,“nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as containing both an ancient grain and an amaranth. If a particular pet food care composition recites both an ancient grain and an amaranth, a compound that may be characterized as both an ancient and an amaranth will serve only as either an ancient or an amaranth—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e. farm cats), pet cats, ferrets, birds, reptiles, rabbits, and fish.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percentage of the ingredient is based on amount of ingredient present as dry matter.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food compositions that increase the weight ratio of propionate to branched short chain fatty acids and, preferably, increase the weight ratio of short chain fatty acids to branched short chain fatty acids in the feces of a pet consuming such pet food compositions. The increase in the weight ratio of propionate to branched short chain fatty acids and short chain fatty acids to branched short chain fatty acids in the feces of the pet was unexpected as the metabolic pathways for producing short chain fatty acids are entangled and, thus, it is difficult to selectively increase specific short chain fatty acids by controlling the diet of a pet. Without being limited to any particular theory, it is believed that the pet food compositions may promote metabolic benefits (e.g., an increase in helpful gut hormones) and reduce anticipatory reward to obesogenic foods, thus regulating appetite and energy homeostasis to reduce obesity, by enhancing the amount of propionate relative to branched short chain fatty acids and short chain fatty acids.

Additionally, it is conventionally believed to be difficult to simultaneously reduce blood circulating uremic solute postbiotics, such as indoles, while concurrently increasing their levels in the colon where such indoles provide benefits to gut immunity. In accordance with certain embodiments, the pet food compositions advantageously increase the amount of indoles in the gut and found in the pet feces while simultaneously reducing the amount of indoles circulating in the blood of the pet.

In accordance with one aspect, provided is a pet food composition having a gut microbiome component that includes β-1,3/1,6-glucan and polyphenolic isoflavonoids, wherein the pet food composition has a weight ratio of β-1,3/1,6-glucan to polyphenolic isoflavonoids of about 1:1 to about 5:1, and wherein the gut microbiome component is present in an amount effective to produce, after about 3 days post-ingestion by a pet, an increase in a weight ratio of propionate to branched short chain fatty acids in the feces and an increase in a weight ratio of short chain fatty acids to branched short chain fatty acids in the feces of the pet.

The microbiome component may be formulated to have a weight ratio of β-1,3/1,6-glucan to polyphenolic isoflavonoids that is about 1:1 to about 4:1. In some embodiments, the weight ratio of β-1,3/1,6-glucan to polyphenolic isoflavonoids is about 1:1 to about 3:1, about 1:1 to about 2:1, about 2:1 to about 5:1, about 3:1 to about 5:1, or about 2:1 to about 4:1. In at least one instance, the pet food composition has a microbiome component with a weight ratio of β-1,3/1,6-glucan to polyphenolic isoflavonoids that is about 2:1 or 2:1.

Preferably, the pet food composition may have a gut microbiome component present in an amount effective to produce, after about 3 days post-ingestion by a pet, a decrease of sulfated indoles in the blood and an increase of indoles in the feces of the pet. In some cases, the pet food composition includes the gut microbiome in an amount from about 0.05 to about 15 wt. %, based on the total weight of the pet food composition on a dry matter basis. For instance, the gut microbiome may be present in the pet food composition in an amount of from about 0.05 to about 12 wt. %, about 0.05 to about 10 wt. %, about 0.05 to about 8 wt. %, about 0.05 to about 6 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.75 wt. %; from about 0.1 to about 15 wt. %, about 0.1 to about 12 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.75 wt. %; from about 0.3 to about 15 wt. %, about 0.3 to about 12 wt. %, about 0.3 to about 10 wt. %, about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %, about 0.3 to about 1 wt. %, about 0.3 to about 0.75 wt. %; from about 0.5 to about 15 wt. %, about 0.5 to about 12 wt. %, about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 0.7 to about 15 wt. %, about 0.7 to about 12 wt. %, about 0.7 to about 10 wt. %, about 0.7 to about 8 wt. %, about 0.7 to about 6 wt. %, about 0.7 to about 5 wt. %, about 0.7 to about 4 wt. %, about 0.7 to about 3 wt. %, about 0.7 to about 2 wt. %; from about 1 to about 15 wt. %, about 1 to about 12 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 1.5 to about 15 wt. %, about 1.5 to about 12 wt. %, about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %; from about 2 to about 15 wt. %, about 2 to about 12 wt. %, about 2 to about 10 wt. %, about 2 to about 8 wt. %, about 2 to about 6 wt. %, about 2 to about 5 wt. %, about 2 to about 4 wt. %, or about 2 to about 3 wt. %, including any range or subrange thereof, based on the total weight of the pet food composition on a dry matter basis.

In some cases, the pet food composition has a concentrated amount of the gut microbiome component. For example, in some embodiments, the gut microbiome component may be present in the pet food composition in an amount of from about 3 to about 15 wt. %, about 3 to about 12 wt. %, about 3 to about 10 wt. %, about 3 to about 8 wt. %, about 3 to about 6 wt. %; from about 5 to about 15 wt. %, about 5 to about 12 wt. %, about 5 to about 10 wt. %, about 5 to about 8 wt. %; from about 7 to about 15 wt. %, about 7 to about 12 wt. %, about 7 to about 10 wt. %; from about 9 to about 15 wt. %, about 9 to about 12 wt. %; from about 11 to about 15 wt. % or about 13 to about 15 wt. %, including ranges and subranges thereof, based on the total weight of the pet food composition on a dry matter basis. A concentrated amount of gut microbiome component may be preferably, for example, when the pet food composition is in the form of a pet food supplement that is adapted to be mixed with a pet food.

The gut microbiome component typically comprises a β-1,3/1,6-glucan (also referred to as (3(1-3;1-6) glucan) and a polyphenolic isoflavonoids. β-1,3/1,6-glucans are compounds comprising a plurality of glucoses that may be linked together in different ways, resulting in different physical and chemical properties. For example, β-1,3/1,6-glucan may have a linear or relatively linear structure, which may provide rheological effects for a pet food composition, such as a thickening effect. The β-1,3/1,6-glucans may also have a plurality of substitutions (e.g., side chains), which may affect the solubility of the β-1,3/1,6-glucans and/or the gut microbiome component in the pet food compositions.

Certain suitable β-1,3/1,6-glucans can be obtained from or derived from yeast, bacteria, fungi and cereal grains as well as other sources. For instance, β-1,3/1,6-glucans can be obtained from or derived from S. cerevisiae, S. delbrueckii, S. rosei, S. microellipsodes, S. carlsbergensis, S. bisporus, S. fermentati, S. rouxii, Schizosaccharomyces pombe, Kluyveromyces polysporus, Candida albicans, C. cloacae, C. tropicalis, C. utilis, Hansenula wingei, H. arni, H. henricii, H. americana, H. canadiensis, H. capsulata, H. polymorpha, Pichia kluyveri, P. pastoris, P. polymorpha, P. rhodanensis, P ohmeri, Torulopsis bovin, and T. glabrata.

β-1,3/1,6-glucans can be in various forms of particulate and/or be in a soluble form in the pet food composition. One example of suitable form of β-1,3/1,6-glucans is whole glucan particle (WGP). The WGP of -1,3/1,6-glucans may be derived from a purified, cell wall preparation obtained from yeast, fungi and cereal grains. The WGPs may have a particle size of about 1 micron or greater. In some cases, it may be desirable to include WGPs of -1,3/1,6-glucans having particle sizes of about 10 microns or more, 100 microns or more, or 500 microns or more.

Another example of a β-1,3/1,6-glucan form is microparticulate glucan particles. Microparticulate glucan particles can be a portions of whole glucan particles that result from finely grinding yeast cell wall β-1,3/1,6-glucan down to a particle size of about 1 micron or less. For instance, the microparticulate glucan particles may have a particle size of about 1 micron or less, about 0.1 micron or less, or about 0.01 micron or less. In yet a further example, the form of β-1,3/1,6-glucans may be neutral soluble β-glucans. Neutral soluble glucans may be prepared through a series of acid, alkaline and neutral treatment steps to yield a conformationally pure neutral soluble glucan preparation.

The gut microbiome component may comprise an amount of β-1,3/1,6-glucan that is from about 0.1 to about 10 wt. %, based on the total weight of the pet food composition on a dry matter basis. For example, the amount of β-1,3/1,6-glucan present in the gut microbiome component may be from about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.6 wt. %; from about 0.2 to about 10 wt. %, about 0.2 to about 8 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.2 to about 1 wt. %, about 0.1 to about 0.8 wt. %, about 0.2 to about 0.6 wt. %; from about 0.3 to about 10 wt. %, about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 0.8 to about 10 wt. %, about 0.8 to about 8 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 6 to about 10 wt. %, or about 6 to about 8 wt. %, including ranges and subranges thereof, based on the total weight of the pet food composition on a dry matter basis.

The gut microbiome component also includes polyphenolic isoflavonoids in an amount that may vary, but typically is in an amount, e.g., from about 0.01 to about 1 wt. %, based on the total weight of the pet food composition. For example, the gut microbiome component may include polyphenolic isoflavonoids in an amount from about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.2 to about 1 wt. %, about 0.2 to about 0.8 wt. %, about 0.2 to about 0.6 wt. %, about 0.2 to about 0.4 wt. %; from about 0.3 to about 1 wt. %, about 0.3 to about 0.8 wt. %, about 0.3 to about 0.6 wt. %, about 0.3 to about 0.4 wt. %; from about 0.5 to about 1 wt. %, or about 0.5 to about 0.8 wt. %, including ranges and subranges thereof, based on the total weight of the pet food composition on a dry matter basis.

The gut microbiome component may comprise various polyphenolic isoflavonoids. Polyphenolic isoflavonoids worth mentioning include, but are not limited to, genistein, daidzein, formononetin, and biochanin A as well as glucosides, acetylglucosides, and malonylglucosides conjugate forms of the isoflavones. Polyphenolic isoflavonoids typically are polyphenolic compounds having a 3-phenylchromen-4-one backbone. In at least one embodiment, the polyphenolic compounds are soy isoflavones, conjugated forms thereof and/or are derived from soy beans. Polyphenolic isoflavonoids can be obtained from legumes, such as soy, chick peas, lentils, beans (broad, haricot, kidney, lima, navy, etc.), grams (Bengal, horse and green) and clovers. Examples of plant species of which polyphenolic isoflavonoids may be obtained or derived from include, e.g., Indian liquorice (Abrus precatorius); various species of Acacia spp. including, A. aneura, A. cibaria, A. longifolia, and A. oswaldii; ground nut (Apio tuberosa); ground pea (Arachis hypogea); milk vetch (Astragalus edulis); marama bean (Bauhinia esculenta); sword bean (Cajanus cajan indicus); jack bean (Canavalia ensiformis); sword bean (Canavalia gladiata); seaside sword bean (Canavalia rosea); various Cassia spp. including C. floribunda, C. laevigata, and C. occidentalis; carobbean (Ceratonia siliqua); chick pea (Cicer arietinum); yebnut (Cordeauxia edulis); various Crotalaria spp. including C. laburnifolia, and C. pallida; cluster bean (Cyamopsis psoralioides); tallow tree (Detariaum senegalense); sword bean (Entada scandens); balu (Erythrina edulis); soyabean (Clycine max;) inga (Ingaedulis); Polynesian chestnut (Inocarpus fagifer); hyacinth bean (Lablab purpureus); grass pea or Indian vetch (Lathyrus sativus); Cyprus vetch (Lathyrus ochrus); lentil (Lens culinaris); jumping bean (Leucaenal eucocephala); various Lupinus spp. including L. albus, L. luteus, L. angustifolium, L. mutabilis, and L. cosentinii; ground bean (Macotylma geocarpa); horse gram (Macrotyloma uniflorum); alfalfa (Medicago sativa); velvet bean (Mucuna pruriens); yam beans (Pachyrhyzuz erosus, P. tuberosus); African locust bean (Parkia clappertoniana); Parkia speciosa; oil bean tree (Pentaclethra macrophylla); various Phaseolus spp. including P. acutifolius, P. vulgaris, P. luntus, P. coccineus, P. adenathus, P. angulris, P. aureus, P. calcaratus, P. mungo, and P. polystachyus; garden pea (Pisum sativum); djenko bean (Pithecolobium lobatum); mesquite (various Prosopis spp.); goa bean (Psophocarpus scandens, P. tetragonolobus); various Psoralea spp.; Sesbania bispinosa; yam bean (Sphenostylis stenocarpa); tamarind (Tamarindus indica); fenugreek (Trigonella foenum-graecum); vetches (various Vivia spp. including V. sativa, V. atropurpurea, V. ervilia, and V. monantha); broad bean (Vicia faba); black gram (Vigna mungo); various Vigna spp. including V. radiata, V. aconitifolia, V. adanatha, V. angularus, V. tribolata, V. umbelata, and V. unguiculata; earth pea (Voandzeia subterranea), etc.

Suitable components, such as those listed below, may be included or excluded from the formulations for the pet food compositions depending on the specific combination of other ingredients and the form of the pet food compositions. In some embodiments, the pet food compositions disclosed herein may be in the form of a standalone pet food, as a supplement to pet food, as a pet food treat, or the like. For example, in some embodiments, the compositions described herein can be used as a dietary supplement and be co-administered with another pet food composition. The dietary supplement can have be in any suitable form, such as a gravy, drinking water, beverage, yogurt, powder, granule, paste, suspension, chew, morsel, treat, snack, pellet, pill, capsule, tablet, sachet, or any other suitable delivery form. The dietary supplement can comprise the dietary formulations and optional compounds such as vitamins, preservatives, probiotics, prebiotics, and antioxidants. This permits the supplement to be administered to the pet in small amounts, or in the alternative, can be diluted before administration.

The pet food compositions may include a prebiotic component. The prebiotic component may comprise at least one prebiotic ingredient and be included in the pet food composition in an amount from about 0.5 to about 20 wt. %, based on the total weight of the pet food composition on a dry matter basis. For example, the pet food composition may contain the prebiotic component or the one or more prebiotic ingredient(s) in an amount from about 0.5 to about 15 wt. %, about 0.5 to about 10 wt. %, about 0.5 to about 8 wt. %, about 0.5 to about 6 wt. %, about 0.5 to about 4 wt. %; from about 1 to about 20 wt. %, about 1 to about 15 wt. %, about 1 to about 10 wt. %, about 1 to about 8 wt. %, about 1 to about 6 wt. %, about 1 to about 4 wt. %; from about 2 to about 20 wt. %, about 2 to about 15 wt. %, about 2 to about 10 wt. %, about 2 to about 8 wt. %, about 2 to about 6 wt. %; from about 4 to about 20 wt. %, about 4 to about 15 wt. %, about 4 to about 10 wt. %, about 4 to about 8 wt. %; from about 6 to about 20 wt. %, about 6 to about 15 wt. %, about 6 to about 10 wt. %; from about 8 to about 20 wt. %, about 8 to about 15 wt. %, about 8 to about 10 wt. %; from about 10 to about 20 wt. %, about 10 to about 15 wt. %; from about 12 to about 20 wt. %, about 12 to about 15 wt. %; from about 14 to about 20 wt. %, or about 16 to about 20 wt. %, based on the total weight of the pet food composition on a dry matter basis.

Prebiotic ingredients are a dietary fiber that generally promote the growth of healthy bacteria having favorable effects on the intestinal flora. The prebiotic component may include one or more prebiotic ingredients selected from fructooligosaccharides (FOS), xylo-oligosaccharides (CBS), galactooligosaccharides (GOS), mannooligosaccharides, and a combination of two or more thereof.

The prebiotic ingredients may be obtained or derived from germinated seeds or grains. The terms “germinated seed” and “sprouted seed” have equivalent meanings. Germinated seeds and grains that are suitable for the pet food composition of the present invention include chia seed, rice, whole grain wheat, oat, buckwheat, broccoli seed, carrot seed, adzuki bean, almond, amaranth, annatto seed, anise seed, arugula, basil, brown rice, navy bean, pinto bean, lima bean, cabbage canola seed, caragana, cauliflower, celery, chick peas, chives, cilantro/coriander/dhania, clover, cress, dill fennel, fenugreek, garlic, hemp, kale, kamut, kat, leek, green lentil, lupins, pearl millet, mizuna, mustard, onion, black-eyed peas, green peas, pigeon peas, snow peas, peanut, psyllium, quinoa, radish, rye, sesame, soybean, tatsoi. triticale, water cress and wheat berries. The prebiotic ingredients may be obtained or derived from other sources as well. For example, fructooligosaccharides (FOS) are naturally found in many foods, such as wheat, onions, bananas, honey, garlic, and leeks.

Additionally or alternatively, the pet food compositions may contain a probiotic component comprising one or more probiotic(s). The probiotic component and/or the one or more probiotics may be included in the pet food composition in an amount from about 0.1 to about 20 wt. %, based on the total weight of the pet food composition on a dry matter basis. For example, the pet food composition may contain a probiotic component and/or one or more probiotics in an amount from about 0.1 to about 15 wt. %, about 0.1 to about 10 wt. %, about 0.1 to about 8 wt. %, about 0.1 to about 6 wt. %, about 0.1 to about 4 wt. %; from about 0.5 to about 20 wt. %, about 0.5 to about 15 wt. %, about 0.5 to about 10 wt. %, about 0.5 to about 8 wt. %, about 0.5 to about 6 wt. %, about 0.5 to about 4 wt. %; from about 1 to about 20 wt. %, about 1 to about 15 wt. %, about 1 to about 10 wt. %, about 1 to about 8 wt. %, about 1 to about 6 wt. %, about 1 to about 4 wt. %; from about 2 to about 20 wt. %, about 2 to about 15 wt. %, about 2 to about 10 wt. %, about 2 to about 8 wt. %, about 2 to about 6 wt. %; from about 4 to about 20 wt. %, about 4 to about 15 wt. %, about 4 to about 10 wt. %, about 4 to about 8 wt. %; from about 6 to about 20 wt. %, about 6 to about 15 wt. %, about 6 to about 10 wt. %; from about 8 to about 20 wt. %, about 8 to about 15 wt. %, about 8 to about 10 wt. %; from about 10 to about 20 wt. %, about 10 to about 15 wt. %; from about 12 to about 20 wt. %, about 12 to about 15 wt. %; from about 14 to about 20 wt. %, or about 16 to about 20 wt. %, based on the total weight of the pet food composition on a dry matter basis.

Typical probiotics include, but are not limited to, probiotic strains selected from Lactobacilli, Bifidobacteria, or Enterococci, e.g., Lactobacillus reuteri, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain is selected from the group consisting of Lactobacillus reuteri (NCC2581; CNCM 1-2448), Lactobacillus reuteri (NCC2592; CNCM 1-2450), Lactobacillus rhamnosus (NCC2583; CNCM 1-2449), Lactobacillus reuteri (NCC2603; CNCM 1-2451), Lactobacillus reuteri (NCC2613; CNCM 1-2452), Lactobacillus acidophilus (NCC2628; CNCM 1-2453), Bifidobacterium adolescentis (e.g., NCC2627), Bificlobacterium sp. NCC2657 or Enterococcus faecium SF68 (NCIMB 10415).

The pet food compositions are formulated to include fat in an amount that may vary, but typically is in the range of about 8 to about 50 wt. %, endpoints included, based on the total weight of the pet food composition on a dry matter basis. For example, the pet food composition may include fat in an amount ranging from about 10 to about 50 wt. %, about 12 to about 50 wt. %, about 14 to about 50 wt. %, about 16 to about 50 wt. %, about 18 to about 50 wt. %, about 20 to about 50 wt. %, about 22 to about 50 wt. %, about 24 to about 50 wt. %; from about 8 to about 40 wt. %, about 10 to about 40 wt. %, about 12 to about 40 wt. %, about 14 to about 40 wt. %, about 16 to about 40 wt. %, about 18 to about 40 wt. %, about 20 to about 40 wt. %, about 22 to about 40 wt. %, about 24 to about 40 wt. %; from about 8 to about 35 wt. %, about 10 to about 35 wt. %, about 12 to about 35 wt. %, about 14 to about 35 wt. %, about 16 to about 35 wt. %, about 18 to about 35 wt. %, about 20 to about 35 wt. %, about 22 to about 35 wt. %, about 24 to about 35 wt. %; about 8 to about 30 wt. %, about 10 to about 30 wt. %, about 12 to about 30 wt. %, about 14 to about 30 wt. %, about 16 to about 30 wt. %, about 18 to about 30 wt. %, about 20 to about 30 wt. %, about 22 to about 30 wt. %, about 24 to about 30 wt. %; from about 8 to about 27 wt. %, about 10 to about 27 wt. %, about 12 to about 27 wt. %, about 14 to about 27 wt. %, about 16 to about 27 wt. %, about 18 to about 27 wt. %, about 20 to about 27 wt. %, about 22 to about 27 wt. %; from about 8 to about 24 wt. %, about 10 to about 24 wt. %, about 12 to about 24 wt. %, about 14 to about 24 wt. %, about 16 to about 24 wt. %, about 18 to about 24 wt. %, about 20 to about 24 wt. %; from about 8 to about 22 wt. %, about 10 to about 22 wt. %, about 12 to about 22 wt. %, about 14 to about 22 wt. %, about 16 to about 22 wt. %, about 18 to about 22 wt. %; from about 8 to about 22 wt. %, about 10 to about 22 wt. %, about 12 to about 22 wt. %, about 14 to about 22 wt. %, about 16 to about 22 wt. %, about 18 to about 22 wt. %; from about 8 to about 20 wt. %, about 10 to about 20 wt. %, about 12 to about 20 wt. %, about 14 to about 20 wt. %, about 16 to about 20 wt. %, including ranges and subranges thereof, based on the total weight of the pet food composition on a dry matter basis.

The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure. The fat may be incorporated completely within the food composition, deposited on the outside of the pet food composition, or a mixture of the two methods. In some embodiments, the pet food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

The fat may comprise dietary fats, such as triglycerides. In some embodiments, the triglyceride may comprise about 20 to about 100%, about 40 to about 100%, about 50 to about 100%, about 60 to about 100%, about 70 to about 100%, about 80 to about 100%, about 90 to about 100%, of the total amount of fat in the pet food composition. In further embodiments, the triglyceride comprises about 20 to about 95%, about 40 to about 95%, about 50 to about 95%, about 60 to about 95%, about 70 to about 95%, about 80 to about 95%, about 90 to about 95%, of the total amount of fat in the pet food composition. In additional embodiments, the triglyceride comprises about 20 to about 90%, about 40 to about 90%, about 50 to about 90%, about 60 to about 90%, about 70 to about 90%, about 80 to about 90%, of the total amount of fat in the pet food composition. In yet further embodiment, the triglyceride comprises about 20 to about 80%, about 40 to about 80%, about 50 to about 80%, about 60 to about 80%, about 70 to about 80%, of the total amount of fat in the pet food composition.

The triglyceride may include one or more constituents that comprise a fatty acid(s) component/moiety. For example, the triglyceride may include one, two, or three aplanatic chains that are selected from fatty acid component. Non-limiting examples of fatty acid components include, but are not limited to, omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof. The fatty acid(s) moieties may be a polyunsaturated fatty acid, such as an omega-3 fatty acid, an omega-6 fatty acid, or a combination of two or more thereof. Non-limiting examples of omega-3 fatty acid components include those selected from linolenic acid, stearidonic acid, eicosatetraenoic acid, eicosapentaenoic acid, docosapentaenoic acid, docosahexaenoic acid, and a combination of two or more thereof. Examples of omega-6 fatty acid moieties include linolenic acid, calendic acid, eicosadienoic acid, arachidonic acid, docosadienoic acid, adrenic acid, osbond acid, tetracosatetraenoic acid, tetracosapentaenoic acid, or a combination of two or more thereof.

The triglyceride may be selected from triglycerides having at least one aliphatic carbon chain comprised of 6 to 10 carbons. For example, the triglyceride may have one, two, or three aliphatic carbons chains of 6 to 10 carbons. In some embodiments, the triglyceride has an aliphatic carbon chain of 6 carbons, 7 carbons, 8 carbons, 9 carbons, and/or 10 carbons. For example, the aliphatic carbon chain of the medium chain triglyceride may include from 6 to 9 carbons, 6 to 8 carbons, 6 or 7 carbons; 7 to 10 carbons, 7 to 9 carbons, 7 or 8 carbons; 8 to 10 carbons, or 8 or 9 carbons, or 9 or 10 carbons. In some embodiments, the triglyceride(s) comprises an aliphatic carbon chain having 6 carbons, 8 carbons, or 10 carbons. For example, the triglyceride may have an aliphatic carbon chain selected from caprylate, caprate, and/or decanoate.

The pet food compositions may optionally contain one or more fatty acid(s). The one or more fatty acids may be selected from monounsaturated fatty acids, polyunsaturated fatty acids, and/or saturated fatty acids. Non-limiting examples of fatty acids include, but are not limited to, omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof. The fatty acid(s) may be a polyunsaturated fatty acid, such as an omega-3 fatty acid, an omega-6 fatty acid, or a combination of two or more thereof. Non-limiting examples of omega-3 fatty acids include those selected from linolenic acid, stearidonic acid, eicosatetraenoic acid, eicosapentaenoic acid, docosapentaenoic acid, docosahexaenoic acid, and a combination of two or more thereof. The pet food composition may include linolenic acid, eicosapentaenoic acid, docosahexaenoic acid, or a combination of two or more thereof. In at least one embodiment, the pet food composition comprises alpha-linolenic acid and/or gamma-linolenic acid. Additionally or alternatively, the polyunsaturated fatty acid may comprise an omega-6 fatty acids. Examples of omega-6 fatty acid include linolenic acid, calendic acid, eicosadienoic acid, arachidonic acid, docosadienoic acid, adrenic acid, osbond acid, tetracosatetraenoic acid, tetracosapentaenoic acid, or a combination of two or more thereof. In some embodiments, the polyunsaturated fatty acid comprises an omega-6 fatty acid selected from linolenic acid, arachidonic acid, and a combination of two or more thereof.

The pet food composition may be formulated to have a weight ratio of omega-3 fatty acids to omega-6 fatty acids of from about 0.5:1 to about 7:1. In some embodiments, the pet food composition has a weight ratio of omega-3 fatty acids to omega-6 fatty acids of from about 0.5:1 to about 6:1, about 0.5:1 to about 5:1, about 0.5:1 to about 4:1, about 0.5:1 to about 3:1, about 0.5:1 to about 2.5:1, about 0.5:1 to about 2:1, about 0.5:1 to about 1.5:1, or about 0.5:1 to about 1:1; from about 1:1 to about 6:1, about 1:1 to about 5:1, about 1:1 to about 4:1, about 1:1 to about 3:1, about 1:1 to about 2.5:1, about 1:1 to about 2:1, about 1:1 to about 1.5:1, or about 1:1 to about 1:1, including ranges or subranges thereof.

The fatty acid(s) present in the pet food composition may be in an amount from about 1 to about 20 wt. %, based on the total weight of the pet food composition on a dry matter basis. For example, the pet food composition may contain fatty acid(s) in an amount from about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 7 to about 20 wt. %, about 7 to about 18 wt. %, about 7 to about 16 wt. %, about 7 to about 14 wt. %, about 7 to about 12 wt. %, about 7 to about 10 wt. %; from about 9 to about 20 wt. %, about 9 to about 18 wt. %, about 9 to about 16 wt. %, about 9 to about 14 wt. %, about 9 to about 12 wt. %; from about 11 to about 20 wt. %, about 11 to about 18 wt. %, about 11 to about 16 wt. %, about 11 to about 14 wt. %; from about 12 to about 20 wt. %, about 12 to about 18 wt. %, about 12 to about 16 wt. %, about 12 to about 14 wt. %; from about 14 to about 20 wt. %, about 14 to about 18 wt. %, or about 14 to about 16 wt. %, including any range or subrange thereof, based on the total weight of the pet food composition on a dry matter basis.

In some cases, the pet food composition may be formulated such that the fatty acids comprise polyunsaturated fatty acid(s) to saturated fatty acid(s) in a weight ratio of from about 7:1 to about 1:7. For example, the weight ratio of the polyunsaturated fatty acid(s) to saturated fatty acid(s) may be from about from about 5:1 to about 1:5, about 3:1 to about 1:3, about 2:1 to about 1:2, or about 1:1. Additionally or alternatively, the pet food compositions may be formulated to have a weight ratio of polyunsaturated fatty acid(s) to monounsaturated fatty acid(s) of about 7:1 to about 1:9. For instance, the pet food compositions may have a weight ratio of unsaturated fatty acid(s) to monounsaturated fatty acid(s) from about 5:1 to about 1:7, about 3:1 to about 1:5, about 2:1 to about 1:4, about 1:1 to about 1:3, or about 1:1 to about 1:2.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

In some cases, the fat in the compositions is crude fat. Crude fat may be included into the compositions in the amounts disclosed above with respect of the total fat, such as from about 8 to about 50 wt. %, based on the total weight of the pet food composition on a dry matter basis. In some embodiments, the pet food composition comprises crude fat in an amount of about 10 to about 40 wt. %, about 12 to about 35 wt. %, about 14 to about 30 wt. %, about 16 to about 24 wt. %, based on the total weight of the pet food composition on a dry matter basis. In some cases, it may be preferable that about 50 wt. % or more, about 60 wt. % or more, about 70 wt. % or more, about 80 wt. % or more, or about 90 wt. % or more of the total fat is obtained from an animal source. Alternatively, about 50 wt. % or more, about 60 wt. % or more, about 70 wt. % or more, about 80 wt. % or more, or about 90 wt. % or more of the total fat may be obtained from a plant source.

The pet food compositions typically include protein in an amount ranging from about 15 to about 55 wt. %, based on the total weight of the pet food composition on a dry matter basis. In some instances, the total amount of protein in the pet food composition is in a range from about 15 to about 50 wt. %, about 15 to about 48 wt. %, about 15 to about 46 wt. %, about 15 to about 44 wt. %, about 15 to about 42 wt. %, about 15 to about 40 wt. %, about 15 to about 38 wt. %, about 15 to about 36 wt. %, about 15 to about 34 wt. %; from about 20 to about 55 wt. %, about 20 to about 50 wt. %, about 20 to about 48 wt. %, about 20 to about 46 wt. %, about 20 to about 44 wt. %, about 20 to about 42 wt. %, about 20 to about 40 wt. %, about 20 to about 38 wt. %, about 20 to about 36 wt. %, about 20 to about 34 wt. %; from about 25 to about 55 wt. %, about 25 to about 50 wt. %, about 25 to about 48 wt. %, about 25 to about 46 wt. %, about 25 to about 44 wt. %, about 25 to about 42 wt. %, about 25 to about 40 wt. %, about 25 to about 38 wt. %, about 25 to about 36 wt. %, about 25 to about 34 wt. %; from about 30 to about 55 wt. %, about 30 to about 50 wt. %, about 30 to about 48 wt. %, about 30 to about 46 wt. %, about 30 to about 44 wt. %, about 30 to about 42 wt. %, about 30 to about 40 wt. %, about 30 to about 38 wt. %, or about 30 to about 36 wt. %, including ranges and subranges therebetween, based on the total weight of the pet food composition on a dry matter basis.

The protein of the pet food composition comprises one or more amino acids selected from Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine, and a combination of two or more thereof. The pet food composition may comprise two or more amino acids. For instance, the pet food composition may include two or more, three or more, four or more, five or more, six or more, seven or more, eight or more amino acids. In some embodiments, the pet food composition includes glycine and proline, and optionally one or more additional amino acids.

In some cases, the one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

A portion of the protein in the composition may be digestible protein. For example, the composition may include an amount of protein, where about 40 wt. % or more, about 50 wt. % or more, about 60 wt. % or more, about 70 wt. % or more, about 80 wt. % or more, about 90 wt. % or more, about 95 wt. % or more, about 98 wt. % or more, or about 99 wt. % or more of the protein is digestible protein. In some embodiments, e.g., when the composition desirable promotes weight loss, the portion of protein that is digestible protein is about 60 wt. % or less, about 50 wt. % or less, about 40 wt. % or less, about 30 wt. % or less, about 20 wt. % or less, or about 10 wt. % or less, based on the total amount of protein in the pet food composition on a dry matter basis. In further embodiment, the amount of protein that is digestible protein is about 10 to about 99 wt. %, about 10 to about 95 wt. %, about 10 to about 90 wt. %, about 10 to about 70 wt. %, about 10 to about 50 wt. %, about 10 to about 30 wt. %; about 30 to about 99 wt. %, about 30 to about 95 wt. %, about 30 to about 90 wt. %, about 30 to about 70 wt. %, about 30 to about 50 wt. %; about 50 to about 99 wt. %, about 50 to about 95 wt. %, about 50 to about 90 wt. %, about 50 to about 70 wt. %; or about 70 to about 99 wt. %, about 70 to about 95 wt. %, about 70 to about 90 wt. %, including ranges and subranges therein, based on the total amount of protein in the pet food composition on a dry matter basis.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The pet food compositions are typically formulated to include fiber in an amount from about 3.5 to about 35 wt. %, based on the total weight of the pet food composition on a dry matter basis. For example, the total amount of fiber present in the pet food composition may be from about 3.5 to about 32 wt. %, about 3.5 to about 29 wt. %, about 3.5 to about 26 wt. %, about 3.5 to about 24 wt. %, about 3.5 to about 22 wt. %, about 3.5 to about 20 wt. %, about 3.5 to about 18 wt. %, about 3.5 to about 16 wt. %; about 5 to about 35 wt. %, about 5 to about 32 wt. %, about 5 to about 29 wt. %, about 5 to about 26 wt. %, about 5 to about 24 wt. %, about 5 to about 22 wt. %, about 5 to about 20 wt. %, about 5 to about 18 wt. %, about 5 to about 16 wt. %; about 8 to about 35 wt. %, about 8 to about 32 wt. %, about 8 to about 29 wt. %, about 8 to about 26 wt. %, about 8 to about 24 wt. %, about 8 to about 22 wt. %, about 8 to about 20 wt. %, about 8 to about 18 wt. %, about 8 to about 16 wt. %; about 11 to about 35 wt. %, about 11 to about 32 wt. %, about 11 to about 29 wt. %, about 11 to about 26 wt. %, about 11 to about 24 wt. %, about 11 to about 22 wt. %, about 11 to about 20 wt. %, about 11 to about 18 wt. %, about 11 to about 16 wt. %; about 14 to about 35 wt. %, about 14 to about 32 wt. %, about 14 to about 29 wt. %, about 14 to about 26 wt. %, about 14 to about 24 wt. %, about 14 to about 22 wt. %, about 14 to about 20 wt. %, about 14 to about 18 wt. %; about 16 to about 35 wt. %, about 16 to about 32 wt. %, about 16 to about 29 wt. %, about 16 to about 26 wt. %, about 16 to about 24 wt. %, about 16 to about 22 wt. %, about 16 to about 20 wt. %; about 18 to about 35 wt. %, about 18 to about 32 wt. %, about 18 to about 29 wt. %, about 18 to about 26 wt. %, about 18 to about 24 wt. %, about 18 to about 22 wt. %; about 20 to about 35 wt. %, about 20 to about 32 wt. %, about 20 to about 29 wt. %, about 20 to about 26 wt. %, about 20 to about 24 wt. %; about 22 to about 35 wt. %, about 22 to about 32 wt. %, about 22 to about 29 wt. %, about 22 to about 26 wt. %; about 24 to about 35 wt. %, about 24 to about 32 wt. %, about 24 to about 29 wt. %; about 26 to about 35 wt. %, about 26 to about 32 wt. %, about 26 to about 29 wt. %; about 28 to about 35 wt. %, or about 28 to about 32 wt. %, including ranges and subranges thereof, based on the total weight of the pet food composition on a dry matter basis.

The total amount of fiber present in the pet food composition generally comprises an amount of crude fiber and dietary fiber. The amount of crude fiber and/or dietary fiber may be present in the pet food compositions in any of the above amounts disclosed for the total amount of fiber. Crude fiber includes indigestible components contained in cell walls and cell contents of plants such as grains, e.g., hulls of grains such as rice, corn, and beans.

Dietary fiber refers to components of a plant that are resistant to digestion by an animal's digestive enzymes. Dietary fiber includes soluble fiber and insoluble fiber. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, cellulose, whole-wheat products, wheat oat, corn bran, flax seed, grapes, celery, green beans, cauliflower, potato skins, fruit skins, vegetable skins, peanut hulls, and soy fiber. Crude fiber includes indigestible components contained in cell walls and cell contents of plants such as grains, for example, hulls of grains such as rice, corn, and beans.

In some embodiments, the pet food composition has a weight ratio of insoluble fiber to soluble fiber from about 20:1 to about 8:1. For example, the pet food composition may have a weight ratio of insoluble fiber to soluble fiber from about 18:1 to about 8:1, about 16:1 to about 8:1, about 14:1 to about 8:1, about 12:1 to about 8:1, about 10:1 to about 8:1. The pet food composition may have a weight ratio of insoluble fiber to soluble fiber of about 11:1.

Additionally and/or alternatively, the fiber component of the pet food composition may comprise an acid detergent fiber and/or a neutral detergent fiber. In some instances, the pet food composition includes an acid detergent fiber and a neutral detergent fiber, e.g., in an amount that is from about 1 to about 20 wt. %, based on the total weight of the pet food composition on a dry matter basis. For example, the pet food composition may include one of an acid detergent fiber and/or a neutral detergent fiber in an amount from about 1 to about 15 wt. %, about 1 to about 10 wt. %, about 1 to about 6 wt. %, about 1 to about 5 wt. %, about 1 to about 3 wt. %; from about 3 to about 15 wt. %, about 3 to about 10 wt. %, about 3 to about 6 wt. %; about 5 to about 15 wt. %, about 5 to about 10 wt. %; about 7 to about 15 wt. %, or about 7 to about 12 wt. %, including ranges and subranges thereof, based on the total weight of the pet food composition on a dry matter basis. In some embodiments, the pet food composition has a weight ratio of the acid detergent fiber to the neutral detergent fiber of from about 3:1 to about 1:3, about 2:1 to about 1:2, or about 1:1.

The pet food composition may further comprise ash. As described herein, ash consists of compounds that are not organic or water, generally produced by combustion of biological materials. The ash may be present in the pet food composition in an amount ranging from about 1 to about 10 wt. %, based on the total weight of the food composition on a dry weight basis, including all amounts and sub-ranges there-between. In some embodiment, the ash may be present in the food composition in an amount ranging from about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the food composition on a dry weight basis.

The pet food composition may include carbohydrates, e.g., in an amount up to about 65 wt. %, based on the total weight of the pet food composition on a dry matter basis. The term “carbohydrate” as used herein includes polysaccharides (e.g., starches and dextrins) and sugars (e.g., sucrose, lactose, maltose, glucose, and fructose) that are metabolized for energy when hydrolyzed. One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, cornstarch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

Generally, carbohydrate percentage can be calculated as nitrogen free extract (“NFE”), which can be calculated as follows: NFE %=100%-(moisture %)-(protein %)-(fat %)-(ash %)-(crude fiber %) The amount of carbohydrate, e.g., calculated as NFE, present in the composition may be from an amount up to about 65 wt. %, an amount up to about 60 wt. %, an amount up to about 55 wt. %, an amount up to about 50 wt. %, an amount up to about 45 wt. %, an amount up to about 40 wt. %, an amount up to about 35 wt. %, an amount up to about 30 wt. %, an amount up to about 25 wt. %, an amount up to about 20 wt. %, an amount up to about 15 wt. %, an amount up to about 10 wt. %, an amount up to about 5 wt. %; about 1 to about 65 wt. %, about 1 to about 55 wt. %, about 1 to about 50 wt. %, about 1 to about 45 wt. %, about 1 to about 40 wt. %, about 1 to about 35 wt. %; about 1 to about 30 wt. %, about 1 to about 25 wt. %, about 1 to about 20 wt. %, about 1 to about 15 wt. %, about 1 to about 10 wt. %, about 1 to about 5 wt. %; about 5 to about 65 wt. %, about 5 to about 55 wt. %, about 5 to about 50 wt. %, about 5 to about 45 wt. %, about 5 to about 40 wt. %, about 5 to about 35 wt. %; about 5 to about 30 wt. %, about 5 to about 25 wt. %, about 5 to about 20 wt. %, about 5 to about 15 wt. %; about 10 to about 65 wt. %, about 10 to about 55 wt. %, about 10 to about 50 wt. %, about 10 to about 45 wt. %, about 10 to about 40 wt. %, about 10 to about 35 wt. %; about 10 to about 30 wt. %, about 10 to about 25 wt. %; about 15 to about 65 wt. %, about 15 to about 55 wt. %, about 15 to about 50 wt. %, about 15 to about 45 wt. %, about 15 to about 40 wt. %, about 15 to about 35 wt. %; about 15 to about 30 wt. %; about 20 to about 65 wt. %, about 20 to about 55 wt. %, about 20 to about 50 wt. %, about 20 to about 45 wt. %, about 20 to about 40 wt. %, about 20 to about 35 wt. %; about 25 to about 65 wt. %, about 25 to about 55 wt. %, about 25 to about 50 wt. %, about 25 to about 45 wt. %, about 25 to about 40 wt. %, about 25 to about 35 wt. %; about 30 to about 65 wt. %, about 30 to about 55 wt. %, about 30 to about 50 wt. %, about 30 to about 45 wt. %; about 35 to about 65 wt. %, about 35 to about 55 wt. %, about 35 to about 50 wt. %; about 40 to about 65 wt. %, about 40 to about 55 wt. %, about 45 to about 65 wt. %, about 45 to about 55 wt. %; or about 50 to about 65 wt. %, including ranges and subranges thereof, based on the total weight of the pet composition on a dry matter basis.

In certain embodiments, the pet food composition comprises moisture. The moisture may be present at various amounts or concentrations. In one embodiment, moisture may be present in an amount of from about 3 to about 20 wt. %, based on the total weight of the pet food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the pet food composition. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

The pet food compositions may include one or more ingredients and/or sources of glucose mimetic, carotenoids, and/or arginine and derivatives thereof. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred embodiment, the pet food composition contains high levels of arginine and derivatives thereof. The amount of arginine present in the composition may be about 0.01 to about 10.0 wt. %, about 0.01 to about 5.0 wt. %, about 0.01 to about 2.0 wt. %, about 0.1 to about 10.0 wt. %, about 0.1 to about 5.0 wt. %, about 0.1 to about 2.0 wt. %; about 0.5 to about 5.0 wt. %, about 0.5 to about 2.0 wt. %, about 1.5 to about 5.0 wt. %, about 1.5 to about 2.0 wt. %, about 0.5 wt. %, about 1.0 wt. %, about 1.4 weight %, about 1.44 wt. %, about 1.8 wt. %, or about 2.0 wt. %, based on the total weight of the composition on a dry matter basis. The arginine present in the composition may be L-arginine, D-arginine, or a mixture thereof.

The pet food compositions disclosed herein may be wet or dry compositions, and the ingredients can be either incorporated into the food composition and/or on the surface of any composition component, such as, for example, by spraying, agglomerating, dusting, or precipitating on the surface. Additionally, the pet food compositions may be formulated and produced to be in various forms and/or consistencies. For instance, the pet food compositions may, for example, be a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles.

The food products may also include components of more than one consistency, for example, soft, chewy meat-like particles or pieces as well as kibble having an outer coating and an inner “core” component. In some embodiments, the pet food compositions may be in the form of a kibble or food kibble. As used herein, the term “kibble” or “food kibble” refers to a particulate pellet, e.g., like a component of feline or canine feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the pet food composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins, minerals, and flavoring. Other additives can include glucosamine, chondroitin sulfate, vegetable extracts, herbal extracts, etc.

The concentration of such additives in the pet food composition typically can be up to about 5 wt. %, based on the total weight of the pet food composition on a dry matter basis. For example, the additives may be present in an amount from about 0.01 to about 5 wt. %, about 0.01 to about 4 wt. %, about 0.01 to about 4 wt. %, about 0.01 to about 3 wt. %, about 0.01 to about 2 wt. %, about 0.01 to about 1 wt. %; about 0.1 to about 5 wt. %, about 0.1 to about 4 wt. %, about 0.1 to about 4 wt. %, about 0.1 to about 3 wt. %, about 0.1 to about 2 wt. %, about 0.1 to about 1 wt. %; about 1 to about 5 wt. %, about 1 to about 4 wt. %, about 1 to about 4 wt. %, about 1 to about 3 wt. %, about 1 to about 2 wt. %; about 2 to about 5 wt. %, about 2 to about 4 wt. %, about 2 to about 4 wt. %, about 2 to about 3 wt. %; about 3 to about 5 wt. %, about 3 to about 4 wt. %; or about 4 to about 5 wt. %, based on the total weight of the pet food composition on a dry matter basis. In some embodiments, the concentration of such additives (particularly where such additives are primarily nutritional balancing agents, such as vitamins and minerals) is from about 0 to about 2.0% by weight, based on the total weight of the pet food composition on a dry matter basis. The amount of additives comprising vitamins may be in addition to the amount of vitamin B discussed above. In some embodiments, the concentration of such additives (again, particularly where such additives are primarily nutritional balancing agents) is from about 0 to about 1.0% by weight, based on the total weight of the pet food composition on a dry matter basis. Although the list of foregoing additives may be potentially suitable in some embodiments, one or more of the foregoing additives may be excluded from other embodiments of the pet food composition.

In specific embodiments, the pet food compositions and food products are formulated to address specific nutritional differences between species and breeds of animals, as well as one of more of the attributes of the animal. For example, cat foods, for example, are typically formulated based upon the life stage, age, size, weight, body composition, and breed.

Sources of proteins, carbohydrates, fats, vitamins, minerals, balancing agents, and the like, suitable for inclusion in the pet food compositions, and particularly in the food products to be administered in methods provided herein, may be selected from among those conventional materials known to those of ordinary skill in the art.

The pet food compositions may include, or in some instance exclude, one or more ingredients selected from beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, keratolytics, egg shell membrane, ancient grain, and a combination of two or more thereof.

The pet food composition may be produced by various methods to achieve the desired pet food composition or desired form for the pet food composition. For example, dry food may be baked or extruded, then cut into individual shaped portions, such as kibbles. In some embodiments, the pet food composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The pet food compositions and food products of the present disclosure can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In accordance with a further aspect of the invention, a method is provided for alleviating, mitigating, and/or reducing renal disease in a pet, the method comprising administering an effective amount of a pet food composition described herein to increase a weight ratio of propionate to branched short chain fatty acids in the blood and an increase in a weight ratio of short chain fatty acids to branched short chain fatty acids in the blood of the pet. The method may include providing and/or feeding the canine a pet food composition disclosed herein. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days. The method may include feeding the pet one time a day, two times a day, three times a day, or in some embodiments four or more times a day.

### EXAMPLES

### Example 1

A non-limiting, exemplary pet food composition (“Example Composition A”) and a comparative pet food composition (“Comparative Composition 1”) were prepared in accordance with aspects of the invention. The formula of Example Composition A and Comparative Composition 1 are shown in Table 1, provided below. Table 2 provides a summary of the nutritional values of Example Composition A and Comparative Composition 1.

TABLE 1

| | Comp. 1 | Ex. A |
|---|---|---|
| | (g/100 g dry | (g/100 g dry |
| Ingredients | matter basis) | matter basis) |
| Whole Corn | 34.2 | 33.4 |
| Rice | 24 | 24 |
| Dried Chicken | 21 | 21 |
| Dried Eggs | 5 | 5 |
| Chicken Fat | 3.1 | 3.1 |
| Cellulose | 3 | 3 |
| Palatant | 4.5 | 4.5 |
| Soybean Oil | 2 | 2 |
| Lactic Acid (84%) | 1.2 | 1.2 |
| Potassium Chloride, Choline Chloride | 1 | 1 |
| (70%), and Sodium Chloride | | |
| Calcium Carbonate and Dicalcium Phosphate | 0.4 | 0.4 |
| Vitamin E (29%) | 0.2 | 0.2 |
| Vitamin Mix | 0.19 | 0.19 |
| Mineral Mix | 0.07 | 0.07 |
| Taurine and Tryptophan | 0.2 | 0.2 |
| Yeast Cell Wall (55% beta-1,3/1,6-glucan) | | 0.2-1 |
| Isoflavones (40%) | | 0.1-0.6 |

TABLE 2

| | Comp. 1 | Ex. A |
|---|---|---|
| | (g/100 g | (g/100 g |
| Nutrient | as fed) | as fed) |
| Moisture | 8.9 | 9.3 |
| Protein Crude | 23.8 | 23.9 |
| Nitrogen Free Extract (NFE) | 46.9 | 46.8 |
| Fat Crude | 13.2 | 13.1 |
| Fiber Crude | 3 | 2.5 |
| Ash | 4.3 | 4.3 |
| Monounsaturated Fatty Acids | 4.7 | 4.8 |
| Polyunsaturated Fatty Acids | 3.7 | 3.7 |
| Saturated Fatty Acids | 3.4 | 3.5 |
| Fiber Insoluble | 7.2 | 7 |
| Fiber Total Dietary | 7.4 | 7 |
| Fiber Soluble | 0.2 | 0.2 |

### Example 2

Example Composition A was evaluated in comparison to Comparative Composition 1 to assess the health effects of Example Composition A. Thirty six canines were fed Comparative Composition 1 for a period of four weeks as a pre-feed regimen. Specifically, nineteen neutered males and seventeen spayed female canines of various ages ranging from 1 to 10 years old and having a mean age of 4.6 years old were evaluated in this study. All of the canines were designated as being healthy. The canines were then randomly separated into two groups, with the first group consuming Example Composition A for a period of four weeks and the second group consuming Comparative Composition 1 for a period of four weeks as a first feed regimen. After the four weeks, the first group of canines was fed Comparative Composition 1 for a period of four weeks and the second group of canines was fed Example Composition A for a period of four weeks as a second feed regimen. After each of the pre-feed regimen, the first feed regimen, and the second feed regimen, blood samples and fecal samples were obtained from each of the canines. The blood samples were analyzed by dependent samples t-test, with the statistical criteria for significance having endpoints set as a=0.05.

All canines were pair-housed in spacious indoor rooms with natural light. The canines received behavioral enrichment by interacting with each other, as well as through play time with caretakers, daily opportunities to run outside, and access to toys. The canines were fed once daily and had ad libitum access to water. All canines were immunized against canine distemper, adenovirus, parvovirus, Bordetella, and rabies, were monitored for parasites, and received routine heartworm preventative.

Example Composition A improved markers associated with gut microbiome and host metabolic support. Notably, levels of the short chain fatty acids (“SCFA”), and specifically propionate, were increased whereas neither acetate nor butyrate were changed (see Table 3, below). Further, two of the three detected branched short chain fatty acids (“BSCFA”) were decreased by Example Composition A.

TABLE 3

| Analyte (ug/g wet | | | |
|---|---|---|---|
| feces, or Ratio) | Comp. 1 | Ex. A | P DIFF* |
| Acetic acid (A) | 4474.3 ± 128.1 | 4384.8 ± 104.9 | 0.55 |
| Propionic acid (P) | 3185.5 ± 135.5 | 3461.8 ± 137.4 | 0.03 |
| Butyric acid (B) | 1848 ± 138.5 | 2180.9 ± 199 | 0.23 |
| 2-Methylbutyric | 127.9 ± 5.9 | 116.7 ± 5.8 | 0.23 |
| acid (2MB) | | | |
| Isobutyric acid (IB) | 206.7 ± 9.2 | 182.6 ± 9.5 | 0.06 |
| Isovaleric acid (IV) | 221.5 ± 10.4 | 198.4 ± 10.7 | 0.08 |
| SCFA (A + P + B) | 9507.9 ± 282.4 | 10027.6 ± 289.5 | 0.12 |
| BSCFA (2MB + IB + | 556.2 ± 24.8 | 497.8 ± 25.4 | 0.09 |
| IV) | | | |
| SCFA/BSCFA | 18.2 ± 0.9 | 21.7 ± 1.1 | 0.02 |
| P/A | 0.71 ± 0.026 | 0.79 ± 0.028 | 5.94E−04 |
| P/SCFA | 0.33 ± 0.009 | 0.34 ± 0.012 | 0.11 |

*P value from dependent samples (paired) t-test

Interestingly, Example Composition A increased the amount of propionate in the canines, while simultaneously decreasing the total amount of BSCFA. Moreover, the increase in the amount of propionate in the canines consuming Example Composition A was the primary driver for the total increase in SCFA. In particular, the ratio of propionate to SCFA and propionate to acetate (“P:A ratio”) increased as well, showing the specificity of Example Composition A to increase propionate more than other SCFA. The overall ratio of SCFA to BSCFA was also increased in canines consuming Example Composition A.

Example Composition A improved gut microbiome markers of gut immune function as shown by the increase of multiple indole ligands of the arylhydrocarbon receptor (“AHR”) in the canines' feces when fed Example Composition A (see Table 4, below). Further, Example Composition A specifically increased multiple AHR-activating indole products (e.g., indolelactate, indoleacetylglycine, oxindoleacetate) of the gut microbiome, while decreasing products of the tryptophanase pathway (e.g., indolin-2-one, hydroxyindolin-2-one).

TABLE 4

| Analyte | | | |
|---|---|---|---|
| (log2 relative | | | |
| fold level) | Comp. 1 | Ex. A | P DIFF* |
| 2-oxindole-3-acetate | −0.23 ± 0.1 | −0.23 ± 0.07 | 0.98 |
| 3-formylindole | −0.02 ± 0.08 | 0.09 ± 0.09 | 0.35 |
| 3-hydroxyindolin-2-one | 0.02 ± 0.11 | −0.17 ± 0.07 | 0.14 |
| 3-indoleglyoxylic acid | 0 ± 0.08 | −0.06 ± 0.06 | 0.36 |
| 5-hydroxyindoleacetate | −0.01 ± 0.15 | −0.21 ± 0.12 | 0.23 |
| indole | −0.11 ± 0.15 | −0.18 ± 0.12 | 0.69 |
| indoleacetate | 0.12 ± 0.08 | −0.03 ± 0.06 | 0.04 |
| indoleacetylglycine | −0.68 ± 0.18 | 0.01 ± 0.16 | 2.5E−04 |
| indolelactate | −0.53 ± 0.37 | 0.85 ± 0.31 | 5.7E−04 |
| indolepropionate | −0.04 ± 0.15 | −0.06 ± 0.21 | 0.92 |
| indolin-2-one | 0.11 ± 0.21 | −0.99 ± 0.26 | 1.2E−04 |
| methyl indole-3-acetate | −0.18 ± 0.13 | −0.17 ± 0.15 | 0.94 |
| oxindolylalanine | −0.2 ± 0.07 | 0.19 ± 0.08 | 1.1E−04 |

*P value from dependent samples (paired) t-test

Additionally, Example Composition A reduced the risk of renal disease as shown by the decrease in amount of 9 out of 20 detected indoles in serum, with none of the 20 indoles being increased in canines consuming Example Composition A (see Table 5, below). The overall decrease in indoles in the blood serum from canines consuming Example Composition A was unexpected and beneficial in view of the increase of indoles in feces (see Table 4). Prominent among the indoles decreased by Example Composition A was indoxyl-3-sulfate, a form of uremic solute associated with renal disease and worsened prognosis for disease progression.

TABLE 5

| Analyte | | | |
|---|---|---|---|
| (log2 relative fold level) | Comp. 1 | Ex. A | P DIFF* |
| 2-oxindole-3-acetate | 0.15 ± 0.19 | −0.08 ± 0.19 | 0.428 |
| 3-formylindole | −0.02 ± 0.06 | −0.1 ± 0.07 | 0.301 |
| 3-hydroxyindolin-2-one | −0.57 ± 0.15 | −0.69 ± 0.15 | 0.463 |
| sulfate | | | |
| 3-indoleglyoxylic acid | −0.04 ± 0.11 | −0.12 ± 0.11 | 0.475 |
| 3-indoxyl sulfate | 0.14 ± 0.08 | −0.18 ± 0.09 | 0.000 |
| 5-hydroxyindole glucuronide | 0.13 ± 0.1 | −0.14 ± 0.1 | 0.008 |
| 5-hydroxyindole sulfate | 0.08 ± 0.08 | −0.3 ± 0.09 | 5.77E−09 |
| 5-hydroxyindoleacetate | 0.02 ± 0.05 | 0.03 ± 0.05 | 0.821 |
| 6-hydroxyindole sulfate | 0.04 ± 0.08 | −0.28 ± 0.1 | 1.11E−04 |
| 7-hydroxyindole sulfate | 0.13 ± 0.1 | −0.07 ± 0.11 | 0.001 |
| indoleacetate | 0.15 ± 0.11 | 0.09 ± 0.14 | 0.778 |
| indoleacetylalanine | −0.27 ± 0.21 | −0.43 ± 0.2 | 0.457 |
| indoleacetylglutamine | 0.09 ± 0.09 | −0.01 ± 0.12 | 0.491 |
| indoleacetylglycine | 0.07 ± 0.1 | −0.04 ± 0.11 | 0.425 |
| indoleacrylate | 0.18 ± 0.19 | −0.21 ± 0.27 | 0.012 |
| indolelactate | −0.11 ± 0.1 | −0.01 ± 0.13 | 0.443 |
| indolepropionate | 0.14 ± 0.18 | −0.27 ± 0.28 | 0.028 |
| indolin-2-one | 0.16 ± 0.1 | −0.22 ± 0.11 | 3.31E−04 |
| indoxyl glucuronide | 0.03 ± 0.13 | −0.12 ± 0.11 | 0.109 |
| oxindolylalanine | 0.04 ± 0.06 | 0.09 ± 0.06 | 0.575 |

*P value from dependent samples (paired) t-test

### Example 3

A non-limiting, exemplary pet food composition (“Example Composition B”) and a comparative pet food composition (“Comparative Composition 2”) were prepared in accordance with aspects of the invention. The formula of Example Composition B and Comparative Composition 2 are shown in Table 6. Table 7 provides a summary of the nutritional values of Example Composition B and Comparative Composition 2.

TABLE 6

| | Comp. 2 | Ex. B |
|---|---|---|
| Corn, yellow, whole | 35.4 | 34.6 |
| Chicken, Dried | 20 | 20 |
| Rice, brewers | 15 | 15 |
| Corn, gluten, meal | 9 | 9 |
| Eggs, dried | 5 | 5 |
| Chicken Fat | 3.1 | 3.1 |
| Cellulose | 3 | 3 |
| Palatant | 3 | 3 |
| Soybean oil | 2 | 2 |
| Lactic acid, 84% | 1.2 | 1.2 |
| Calcium sulfate | 1 | 1 |
| Potassium chloride | 1 | 1 |
| Sodium chloride, iodized | 0.3 | 0.3 |
| Taurine | 0.25 | 0.25 |
| Choline chloride, 70% | 0.23 | 0.23 |
| Vitamin E, 29% | 0.2 | 0.2 |
| Vitamin, premix | 0.19 | 0.19 |
| Tryptophan | 0.07 | 0.07 |
| Mineral, premix | 0.07 | 0.07 |
| Yeast Cell Wall (55% BG) | | 0.5 |
| Isoflavone (40% isoflavonoids) | | 0.3 |

TABLE 7

| | Comp. 2 | Ex. B | Percent Difference |
|---|---|---|---|
| | (wt. % on dry | (wt. % on dry | ((Ex. B − Comp. |
| Analyte | matter basis) | matter basis) | 2)/Comp Ex 1)*100 |
| Ash | 5.49 | 5.34 | −2.7 |
| Fat Crude | 13.24 | 13.94 | 5.3 |
| Fiber Crude | 2.8 | 3 | 7.1 |
| Protein Crude | 28.63 | 28.5 | −0.5 |
| Starch | 35.3 | 36.1 | 2.3 |
| Sugars - Total | 0.73 | 0.74 | 1.4 |

### Example 4

Example Composition B was evaluated in comparison to Comparative Composition 2 to assess the health effects of Example Composition B. Twenty felines were fed Comparative Composition 2 for a period of four weeks as a pre-feed regimen. All of the felines were designated as being healthy. The felines were then randomly separated into two groups, with the first group consuming Example Composition B for a period of four weeks and the second group consuming Comparative Composition 2 for a period of four weeks as a first feed regimen.

After the four weeks, the first group of canines was fed Comparative Composition 2 for a period of four weeks and the second group of canines was fed Example Composition B for a period of four weeks as a second feed regimen. After each of the pre-feed regimen, the first feed regimen, and the second feed regimen, blood samples and fecal samples were obtained from each of the felines. The blood samples were analyzed by dependent samples t-test, with the statistical criteria for significance having endpoints set as a=0.05.

All felines were pair-housed in spacious indoor rooms with natural light. The felines received behavioral enrichment by interacting with each other, as well as through play time with caretakers, daily opportunities to run outside, and access to toys. The felines were provided food once daily with access to such food for 22 hours per day and had ad libitum access to water. All felines were immunized.

A summary of the polyphenols from the feces analysis is provided in Table 8.

TABLE 8

| | | | Difference | |
|---|---|---|---|---|
| | | | (Ex. B − | Prob > |
| Metabolite | Comp. 2 | Ex. B | Comp. 2) | \|t\| |
| glycitein | −2.51 ± 0.25 | 4.84 ± 0.17 | 7.35 | 3.25E−34 |
| daidzein | −0.95 ± 0.16 | 4.34 ± 0.18 | 5.29 | 7.09E−32 |
| genistein | −1.12 ± 0.21 | 4.75 ± 0.22 | 5.87 | 7.08E−29 |
| genistein sulfate* | −0.45 ± 0.33 | 8.18 ± 0.29 | 8.63 | 1.71E−28 |
| glycitein sulfate (2) | −4.83 ± 0.37 | 4.32 ± 0.34 | 9.15 | 7.01E−27 |
| daidzein sulfate | −1.31 ± 0.4 | 7.52 ± 0.29 | 8.83 | 1.18E−26 |
| equol sulfate | −0.96 ± 0.25 | 7.57 ± 0.49 | 8.53 | 2.09E−23 |
| equol | −5.95 ± 0.27 | 3.17 ± 0.53 | 9.12 | 3.8E−23 |
| hesperetin | 1.48 ± 0.18 | −0.63 ± 0.14 | −2.11 | 1.27E−13 |
| hesperidin (hesperetin 7-rutinoside) | 1.28 ± 0.17 | −1.03 ± 0.19 | −2.31 | 5.74E−13 |
| ponciretin | 1.11 ± 0.18 | −1 ± 0.15 | −2.11 | 8.86E−13 |
| oleanolate | −1.73 ± 0.21 | −3.95 ± 0.16 | −2.22 | 4.32E−12 |
| limonin | −0.39 ± 0.25 | −2.65 ± 0.13 | −2.26 | 2.64E−11 |
| eriodictyol | 0.35 ± 0.24 | −1.43 ± 0.23 | −1.78 | 1.45E−06 |
| secoisolariciresinol | 0.9 ± 0.26 | −0.74 ± 0.22 | −1.64 | 8.16E−06 |
| enterodiol | −0.78 ± 0.45 | −3.56 ± 0.42 | −2.78 | 2.51E−05 |
| chrysoeriol (3′-O-methylluteolin) | 0.22 ± 0.19 | −0.84 ± 0.19 | −1.06 | 0.0002 |
| ferulate | 0.01 ± 0.11 | −0.61 ± 0.12 | −0.62 | 0.0002 |
| enterolactone | −2.17 ± 0.34 | −3.88 ± 0.3 | −1.71 | 0.0003 |
| dihydrocaffeate sulfate (2) | −0.76 ± 0.32 | 0.44 ± 0.18 | 1.2 | 0.0018 |
| naringenin | 0.06 ± 0.17 | 0.85 ± 0.19 | 0.79 | 0.0023 |
| enterolactone sulfate | 0.32 ± 0.25 | −0.61 ± 0.2 | −0.93 | 0.005 |
| dihydroferulate | 0.26 ± 0.17 | −0.33 ± 0.14 | −0.59 | 0.01 |
| cinnamoylglycine | −0.16 ± 0.26 | 0.61 ± 0.21 | 0.77 | 0.03 |
| vanillin sulfate | −0.19 ± 0.41 | 0.68 ± 0.31 | 0.87 | 0.1 |
| vanillic acid glycine | −0.03 ± 0.14 | 0.27 ± 0.12 | 0.3 | 0.11 |
| vanillate | 0.13 ± 0.12 | −0.12 ± 0.12 | −0.25 | 0.17 |
| syringic acid | −0.03 ± 0.17 | −0.29 ± 0.14 | −0.26 | 0.25 |
| dihydroferulic acid sulfate | 0.23 ± 0.43 | 0.76 ± 0.28 | 0.53 | 0.31 |
| caffeic acid sulfate | −0.53 ± 0.39 | −0.04 ± 0.3 | 0.49 | 0.33 |
| caffeate | −0.14 ± 0.19 | 0.09 ± 0.15 | 0.23 | 0.34 |
| 4-hydroxycinnamate | 0.39 ± 0.12 | 0.24 ± 0.13 | −0.15 | 0.38 |
| sinapate | 0.3 ± 0.13 | 0.13 ± 0.15 | −0.17 | 0.39 |
| deoxymugineic acid | 0.19 ± 0.09 | 0.1 ± 0.07 | −0.09 | 0.42 |
| apigenin | −0.16 ± 0.29 | 0.58 ± 0.46 | −0.42 | 0.44 |
| pheophorbide A | −1.26 ± 0.04 | −1.31 ± 0.04 | −0.05 | 0.44 |
| feruloylputrescine | 0.23 ± 0.24 | 0.01 ± 0.26 | −0.22 | 0.52 |
| DIMBOA | −0.34 ± 0.13 | −0.27 ± 0.09 | 0.07 | 0.66 |
| 3-dehydroshikimate | 0.2 ± 0.28 | 0.35 ± 0.25 | 0.15 | 0.68 |
| quinate | 0.78 ± 0.24 | 0.65 ± 0.23 | −0.13 | 0.7 |
| furaneol sulfate | 0.27 ± 0.25 | 0.18 ± 0.19 | −0.09 | 0.78 |
| dehydroquinate | −0.51 ± 0.11 | −0.49 ± 0.11 | 0.02 | 0.91 |
| ferulic acid 4-sulfate | 0.55 ± 0.52 | 0.61 ± 0.5 | 0.06 | 0.93 |

*designates metabolites for which identification was made without an identical internal standard by characterization of the molecular weight and mass spec fragmentation pattern.
**designates metabolites for which a compound type/class can be determined, although the entire molecular structure was not determined.
(2) designates an alternate form of a metabolite having a given name.

Example Composition B increased the amount of isoflavonoids (e.g., glycitein, daidzein, genistein, genistein sulfate*, glycitein sulfate (2), daidzein sulfate, equol sulfate, and equol) in the feces and decreased the amount of non-isoflavonoids polyphenols. It was unexpectedly determined that Example Composition B decreased the amount of fecal plant flavonoids molecules even though there was not enough increase in the isoflavonoids-containing ingredient in Example Composition B versus Comparative Composition 2 (e.g. corn and rice) to explain the meaningful decrease in the level of plant flavonoids, as seen in Table 6. For instance, the plant flavonoids in Example Composition B as compared to Comparative Composition 2 decreased even though there were the same amount of them in both diets. In contrast, Example Composition B yielded much more isoflavonoids than Comparative Composition 2. This indicates that the isoflavonoids were decreasing flavonoids not by a biological mechanism, not as a result of flavonoids being replaced by isoflavonoids in the formulation of Example Composition B as compared to Comparative Composition 2.

A summary of the amino acids from the feces analysis is presented in Table 9.

TABLE 9

| | | | Difference | |
|---|---|---|---|---|
| | | | (Ex. B − | Prob > |
| Amino Acid | Comp. 2 | Ex. B | Comp. 2) | \|t\| |
| leucine | 0.04 ± 0.09 | −0.48 ± 0.06 | −0.52 | 1.4E−05 |
| isoleucine | 0.1 ± 0.1 | −0.4 ± 0.06 | −0.5 | 3.9E−05 |
| valine | 0.18 ± 0.1 | −0.36 ± 0.07 | −0.54 | 5.3E−05 |
| methionine | −0.12 ± 0.11 | −0.62 ± 0.08 | −0.5 | 3.0E−04 |
| serine | 0.06 ± 0.13 | −0.59 ± 0.11 | −0.65 | 3.1E−04 |
| phenylalanine | −0.07 ± 0.1 | −0.52 ± 0.06 | −0.45 | 4.3E−04 |
| tyrosine | −0.04 ± 0.11 | −0.53 ± 0.09 | −0.49 | 0.001 |
| alanine | 0.08 ± 0.1 | −0.35 ± 0.09 | −0.43 | 0.002 |
| glutamine | −0.1 ± 0.12 | −0.55 ± 0.1 | −0.45 | 0.006 |
| threonine | 0.19 ± 0.08 | −0.12 ± 0.07 | −0.31 | 0.007 |
| asparagine | −0.12 ± 0.17 | −0.74 ± 0.17 | −0.62 | 0.013 |
| cysteine | 0.08 ± 0.12 | 0.46 ± 0.14 | 0.38 | 0.052 |
| aspartate | 0.17 ± 0.17 | 0.43 ± 0.16 | 0.26 | 0.263 |
| proline | 0.16 ± 0.1 | 0.02 ± 0.11 | −0.14 | 0.332 |
| glycine | 0.38 ± 0.14 | 0.21 ± 0.14 | −0.17 | 0.385 |
| cystine | 0.36 ± 0.17 | 0.16 ± 0.26 | −0.2 | 0.516 |
| lysine | −0.06 ± 0.12 | −0.15 ± 0.1 | −0.09 | 0.586 |
| arginine | 0.8 ± 0.26 | 0.9 ± 0.24 | 0.1 | 0.785 |
| tryptophan | 0.16 ± 0.12 | 0.12 ± 0.13 | −0.04 | 0.788 |
| histidine | 0 ± 0.12 | −0.04 ± 0.15 | −0.04 | 0.827 |
| taurine | 0.12 ± 0.14 | 0.16 ± 0.09 | 0.04 | 0.839 |
| glutamate | 0.03 ± 0.09 | 0.05 ± 0.07 | 0.02 | 0.912 |

Example Composition B decreased the amount of fecal proteolytic amino acids, in comparison to Comparative Composition 2, which was surprising as the protein digestability of Example Composition B and Comparative Composition 2 was substantially the same (95.4% protein digestability for Example Composition B and 96% protein digestability for Comparative Ex 1). Thus, the decrease in the fecal amino acids and apparent microbial proteolysis in the felines being fed Example Composition B was not due to changes in the overall protein digestibility, but was due to a direct effect of Example Composition B on microbial metabolic propensity. A reduction in fecal amino acids is indicative of decreased gut microbiome proteolysis as amino acids are typically generated in the colon by microbial breakdown of dietary protein that bypassed digestion.

A summary of the urobilins from the feces analysis is presented in Table 10. As seen in Table 10, Example Composition B decreased the amount of haemolytic urobilins in the felines' feces.

TABLE 10

| | | | Difference | |
|---|---|---|---|---|
| | | | (Ex. B − | Prob > |
| Metabolite | Comp. 2 | Ex. B | Comp. 2) | \|t\| |
| bilirubin degradation product, | 0.67 ± 0.1 | −0.15 ± 0.13 | −0.82 | 4.5E−06 |
| C17H18N2O4 (2)** | | | | |
| bilirubin degradation product, | 0.83 ± 0.11 | −0.002 ± 0.13 | −0.83 | 6.6E−06 |
| C17H18N2O4 (3)** | | | | |
| biliverdin | 0.29 ± 0.06 | −0.13 ± 0.07 | −0.42 | 3.1E−05 |
| bilirubin degradation product, | 0.27 ± 0.17 | −0.8 ± 0.18 | −1.07 | 4.6E−05 |
| C17H18N2O4 (1)** | | | | |
| bilirubin (Z,Z) | 0.43 ± 0.12 | −0.07 ± 0.13 | −0.50 | 0.007 |
| bilirubin degradation product, | 0.14 ± 0.12 | −0.35 ± 0.14 | −0.49 | 0.009 |
| C16H18N2O5 (2)** | | | | |
| L-urobilin | −1.07 ± 0.43 | −2.13 ± 0.36 | −1.06 | 0.064 |
| D-urobilin | 0.24 ± 0.22 | −0.25 ± 0.17 | −0.49 | 0.084 |
| bilirubin (E,E)* | 0.15 ± 0.09 | −0.01 ± 0.12 | −0.16 | 0.316 |
| bilirubin degradation product, | 0.08 ± 0.11 | 0.1 ± 0.1 | −0.08 | 0.621 |
| C16H18N2O5 (1)** | | | | |
| I-urobilinogen | −0.41 ± 0.35 | −0.27 ± 0.27 | 0.14 | 0.751 |

*designates metabolites for which identification was made without an identical internal standard by characterization of the molecular weight and mass spec fragmentation pattern
**designates metabolites for which a compound type/class can be determined, although the entire molecular structure was not determined.
(1)-(3) designates an alternate form of a metabolite having a given name.

A summary of the ethanolamide to acylglycerol ratios from the feces analysis is presented in Table 11.

TABLE 11

| | | | Difference | |
|---|---|---|---|---|
| | | | (Ex. B − | Prob > |
| Metabolite | Comp. 2 | Ex. B | Comp. 2) | \|t\| |
| Oleoylethanolamide to | 0.35 ± 0.1 | 0.82 ± 0.1 | 0.47 | 0.002 |
| oleoylglycerol ratio | | | | |
| Palmitoleoylethanolamide to | 0.19 ± 0.09 | 0.61 ± 0.07 | 0.42 | 6.0E−04 |
| palmitoleoylglycerol ratio | | | | |
| Palmitoylethanolamide to | 0.22 ± 0.1 | 0.49 ± 0.08 | 0.27 | 0.036 |
| palmitoylglycerol ratio | | | | |

1. A pet food composition comprising:
a gut microbiome component comprising:
(i) β-1,3/1,6-glucan;
(ii) polyphenolic isoflavonoids,
wherein pet food composition has a weight ratio of β-1,3/1,6-glucan (i) to polyphenolic isoflavonoids (ii) of about 1:1 to about 5:1,
wherein the gut microbiome component is present in an amount effective to produce, after about 3 days post-ingestion by a pet, an increase in a weight ratio of propionate to branched short chain fatty acids in the feces and an increase in a weight ratio of short chain fatty acids to branched short chain fatty acids in the feces of the pet.

2. The pet food composition according to claim 1, wherein the weight ratio of β-1,3/1,6-glucan (i) to polyphenolic isoflavonoids (ii) is about 1:1 to about 3:1.

3. The pet food composition according to claim 1, wherein the weight ratio of β-1,3/1,6-glucan (i) to polyphenolic isoflavonoids (ii) is about 2:1.

4. The pet food composition according to claim 1, wherein the gut microbiome component is present in an amount effective to produce, after about 3 days post-ingestion by a pet, a decrease of sulfated indoles in the blood and an increase of indoles in the feces of the pet.

5. The pet food composition according to claim 1, further comprising a prebiotic component.

6. The pet food composition according to claim 5, wherein the prebiotic component comprises beet pulp, citrus pulp, or a mixture thereof.

7. The pet food composition according to claim 1, wherein the prebiotic component comprises one or more germinated seeds wherein the one or more geminated seed is selected from chia seed, rice, whole grain wheat, oat, buckwheat, broccoli seed, carrot seed, adzuki bean, almond, amaranth, annatto seed, anise seed, arugula, basil, brown rice, navy bean, pinto bean, lima bean, cabbage canola seed, caragana, cauliflower, celery, chick peas, chives, cilantro/coriander/dhania, clover, cress, dill fennel, fenugreek, garlic, hemp, kale, kamut, kat, leek, green lentil, lupins, pearl millet, mizuna, mustard, onion, black-eyed peas, green peas, pigeon peas, snow peas, peanut, psyllium, quinoa, radish, rye, sesame, soybean, tatsoi. triticale, water cress, wheat berries, and a combination of two or more thereof.

8. (canceled)

9. The pet food composition according to claim 1, wherein the β-1,3/1,6-glucan is present in an amount of from about 0.1 to about 10 wt. %, based on the total weight of the pet food composition.

10. The pet food composition according to claim 1, wherein the amount of β-1,3/1,6-glucan is from about 0.3 to about 5 wt. %, based on the total weight of the pet food composition.

11. The pet food composition according to claim 1, wherein the polyphenolic isoflavonoids is present in an amount of from about 0.01 to about 1 wt. %, based on the total weight of the pet food composition.

12. The pet food composition according to claim 1, wherein the amount of polyphenolic isoflavonoids is from about 0.05 to about 0.5, based on the total weight of the pet food composition.

13. A pet food composition comprising:
a prebiotic component; and
a gut microbiome component comprising:
(i) β-1,3/1,6-glucan;
(ii) polyphenolic isoflavonoids,
wherein pet food composition has a weight ratio of β-1,3/1,6-glucan (i) to polyphenolic isoflavonoids (ii) of about 1:1 to about 5:1.

14. The pet food composition according to claim 13, wherein the β-1,3/1,6-glucan is present in an amount of from about 0.3 to about 5 wt. %, based on the total weight of the pet food composition.

15. The pet food composition according to claim 13, wherein the polyphenolic isoflavonoids is present in an amount of from about 0.05 to about 0.5, based on the total weight of the pet food composition.

16. The pet food composition according to claim 13, wherein the weight ratio of β-1,3/1,6-glucan to polyphenolic isoflavonoids is about 1:1 to about 3:1.

17. (canceled)

18. The pet food composition according to claim 13, wherein the pet food composition has a weight ratio of polyunsaturated fatty acid(s) to saturated fatty acid(s) of from about 7:1 to about 1:7.

19. The pet food composition according to claim 13, wherein the pet food composition has a weight ratio of polyunsaturated fatty acid(s) to monounsaturated fatty acid(s) of about 7:1 to about 1:9.

20. A method for alleviating, mitigating, and/or reducing renal disease in a pet, the method comprising: administering an effective amount of a pet food composition according to claim 13 to increase a weight ratio of propionate to branched short chain fatty acids in the blood and an increase in a weight ratio of short chain fatty acids to branched short chain fatty acids in the blood of the pet.

21. The method according to claim 20, wherein the canine is fed the pet food composition at least once a day, preferably at least twice a day, or preferably at least three times a day.

22. The method according to claim 20, wherein the canine is fed the pet food composition for 1 or more days, preferably 5 or more days, preferably 7 or more days, preferably 10 or more days, preferably 14 or more days, preferably 30 or more days, or preferably 42 or more days.
